(19) United States
(12) Patent Application Publication
(10) Pub. No.: US 2020/0074313 A1
(43) Pub. Date: Mar. 5, 2020

(54) DETERMINING FEATURES TO BE INCLUDED IN A RISK ASSESSMENT INSTRUMENT

(71) Applicant: KONINKLIJKE PHILIPS N.V., EINDHOVEN (NL)

(72) Inventors: Reza Sharifi Sedeh, Malden, MA (US); Gabriel Ryan Mankovich, Boston, MA (US); Hans-Aloys Wischmann, Henstedt-Ulzburg (DE)

(21) Appl. No.: 16/547,880

(22) Filed: Aug. 22, 2019

Related U.S. Application Data
(60) Provisional application No. 62/724,112, filed on Aug. 29, 2018.

(51) Int. Cl. G06N 5/02

(52) U.S. Cl. CPC G06N 5/02; G16H 50/30

(57) ABSTRACT

The invention discloses an apparatus for determining features to be included in a risk assessment instrument. The apparatus comprises a processor configured to: receive an indication of a plurality of features to be analyzed, each feature of the plurality of features being potentially relevant to a likelihood of the presence of a defined target condition; apply, using the plurality of features, a predictive model to a dataset representing the ground truth in relation to the defined target condition; determine, based on an output of the predictive model, one or more features of the plurality of features that are most relevant to the likelihood of the presence of the defined target condition; and determine, based on an output of the predictive model, a threshold value for each of the one or more features, beyond which the likelihood of the presence of the defined target condition is increased or decreased. A method and a computer program product are also disclosed.

### RELATED APPLICATION

This application claims priority to and the benefit of U.S. Provisional Application Ser. No. 62/724,112, filed Aug. 29, 2018, which is incorporated by reference herein in its entirety.

### FIELD OF THE INVENTION

The invention relates to risk assessment instruments and, more particularly, to an apparatus and method for determining features to be included in a risk assessment instrument.

### BACKGROUND OF THE INVENTION

Risk assessment instruments (also referred to as risk scoring assessments) provide a relatively straightforward mechanism by which a user can determine a risk score, or likelihood score, for a particular condition. A risk assessment instrument typically includes a plurality of questions or options, and a score is allocated based on the answer to the question or the option selected. Based on the total score (obtained, for example, by adding, subtracting and/or multiplying the individual scores), a user is able to determine a risk or likelihood that a particular condition is present, has occurred or will occur. Risk assessment instruments are generally easy to understand, and provide users with insights into models used to determine the risk score, without the user needing an in-depth knowledge or understanding of the subject at hand. Risk assessment instruments provide an efficient and effective means for objectively underpinning a qualitative assessment of a situation or status by an expert, for aiding a non-expert to quickly make an assessment, for enabling such assessments to be repeated frequently to enable detection of potentially subtle changes in status that might otherwise go unnoticed.

Risk assessment instruments are used in various environments, particularly where high-stake decisions are to be made. For example, risk assessment instruments are used in clinical settings to enable clinicians to assess the risk of a patient suffering from a particular medical condition. In a different setting, risk assessment instruments may also be used to calculate the risk that a convicted criminal will reoffend and in determining the sentence to be given to an offender.

Each risk assessment instrument is configured to calculate a risk score based on a set of features, the features being specific to the risk being calculated. For example, in a clinical setting, a risk assessment instrument may include features such as a patient's age or gender, a measurement taken in respect of the patient (e.g. blood pressure) or a measurement taken from a medical image captured in respect of the patient (e.g. a size or volume of an anatomical object).

In some cases, particularly in healthcare, designers of risk assessment instruments strive for instruments that are: (i) practical (e.g. the user of the instrument should be able to assess the risk using simple calculations), (ii) transparent (e.g. the user should be able to see generally how the model functions, and which features are used), and (iii) interpretable (e.g. the user should be able to make sense of the model, and the model should observe constraints defined by domain experts).

Existing risk assessment instruments tend to be designed based either on knowledge-based techniques (i.e. a panel of experts creates a scoring system based on their domain knowledge) or data-driven techniques (i.e. machine learning models are used to create risk scoring systems). Inappropriately designing or simplifying such models can result in a model that is not interpretable by the user and/or that exhibits poor and/or unpredictable performance.

One way to improve the interpretability of a risk assessment instrument for a user is to present features to be taken into account by the instrument in a binarized form, in which each feature is presented in such a way that a user has two possible answers (e.g. “yes” or “no”) or options. Thus, as used herein, “binarizing” will be understood as meaning representing something in binary form. Presenting features in a binary manner helps users to better understand the model and the particular features that contribute to the risk being assessed. For example, questions based on features (e.g. age and hippocampus volume) for early detection of Alzheimer's disease might be:

1) Is Age less than 65?

2) Is Age greater than or equal to 65?

3) Is Age greater than or equal to 70?

4) Is Hippocampus' volume less than 2.5 cubic centimeters?

In such an example, a different integer score may be assigned to each answer to each question (or feature). If the answer to a question is “no”, then no integer score is assigned for that question. If the answer to a question is “yes”, then the corresponding integer score is added to a total score for the subject using the risk assessment instrument.

The selection of the above-mentioned thresholds (e.g., 65 and 70 for age, and 2.5 for the hippocampus' volume) is ad hoc. One way to define these thresholds is to use domain knowledge. For example, it is understood that people aged under 65 have a relatively lower risk of developing Alzheimer's disease, which leads to the first ‘age’ threshold of 65. Alternatively, a data-driven approach to define the thresholds may be taken. For example, the first quartile (25%), the median (50%) and the third quartile (75%) of the age distribution of a particular cohort may be used as three thresholds to be considered, such that the feature (i.e. age) is presented in a binarized form based on the three thresholds.

When determining the features and/or the thresholds to use in a risk assessment instrument, designers of such an instrument may take into account the number of thresholds to be included for each feature or variable (e.g. age/hippocampus volume), the values to be used as the thresholds, and whether knowledge-based or data-driven techniques should be used to define the thresholds. Arbitrarily choosing to use a knowledge-based threshold (e.g. age ≥65 as an indicator of an increased risk of the onset of Alzheimer's disease) may lead to the omission of other relevant thresholds which, for example, may be obtained from a data-driven approach (e.g. using as a threshold a tercile, a quartile or a quintile of a frequency distribution of the age of patients suffering from Alzheimer's disease).

Therefore, it would be useful to have a system in which the designer of such an instrument need not select a priori a particular individual threshold to include when assessing a risk, when doing so might omit some other potentially more relevant threshold.

### SUMMARY OF THE INVENTION

It has been recognized by the inventors of the present disclosure that a more accurate risk assessment instrument may be generated when a greater number of thresholds are considered for features to be included in the instrument. In particular, it has been recognized that, by implementing a computer model (e.g. a predictive model such as a machine learning model), many features and thresholds may be considered, and those features and thresholds determined to be most relevant to the risk under consideration (e.g. those features and thresholds that are most relevant to population of subjects) may be selected for inclusion in a risk assessment instrument.

According to a first aspect, various embodiments provide an apparatus for determining features to be included in a risk assessment instrument, the apparatus comprising: a processor configured to: receive an indication of a plurality of features to be analyzed, each feature of the plurality of features being potentially relevant to a likelihood of the presence of a defined target condition; apply, using the plurality of features, a predictive model to a dataset representing the ground truth in relation to the defined target condition; determine, based on an output of the predictive model, one or more features of the plurality of features that are most relevant to the likelihood of the presence of the defined target condition; and determine, based on an output of the predictive model, a threshold value for each of the one or more features, beyond which the likelihood of the presence of the defined target condition is increased or decreased.

In this way, the most relevant features and corresponding threshold values for inclusion in a risk assessment instrument can be determined, rather than the features and thresholds being predetermined, or determined in an arbitrary manner. By using a predictive model to determine the most relevant features based on a set of data representing the ground truth, various options of features and thresholds can be taken into account and considered, and those features whose use results in the predictive model performing the best on the ground truth cohort can be selected to be included in the risk assessment instrument. In this way, the resulting risk assessment instrument provides a more accurate measure of the likelihood of the presence of the target condition. In a clinical setting, an accurate risk assessment instrument can result in a more reliable diagnosis of a subject and, consequently, a more positive health outcome for the subject.

In some embodiments, the determined threshold value may comprise a threshold value selected from a group of threshold values including: a quantile of a frequency distribution of the dataset; and a user-defined threshold.

The processor may, in some embodiments, be further configured to: generate a risk assessment instrument interface including the determined one or more features and the determined threshold value for each of the determined one or more features.

The predictive model may comprise a logistic regression model.

In some embodiments, the predictive model applied to the dataset may be parameterized as an integer programming problem.

The processor may, in some embodiments, be configured to: calculate a measure according to a quality metric for the output of the predictive model. The processor may be configured, responsive to determining that the calculated quality measure is below a defined quality measure threshold, to: add a feature to the plurality of features to obtain a revised feature set; apply, using the revised feature set, the predictive model to the dataset; determine, based on an output of the predictive model, one or more features of the revised feature set that are most relevant to the likelihood of the presence of the defined target condition; determine, based on an output of the predictive model, a threshold value for each of the one or more features, beyond which the likelihood of the presence of the defined target condition is increased or decreased; and determine a revised quality measure according to the quality metric for the output of the predictive model.

In some embodiments, the apparatus may further comprise a user interface for receiving from a user the indication of the plurality of features to be analyzed and/or an indication of a plurality of threshold values from which the threshold value for each of the one or more features is to be determined.

The user interface may further be for presenting to a user an indication of the determined one or more features and an indication of the determined threshold for each of the one or more features. The user interface may further be for presenting to a user a graphical representation of the determined one or more features and an indication of the determined threshold for each of the one or more features. Thus, the indication may comprise a graphical representation.

According to a second aspect, various embodiments provide a method for determining features to be included in a risk assessment instrument, the method comprising: receiving an indication of a plurality of features to be analyzed, each feature of the plurality of features being potentially relevant to a likelihood of the presence of a defined target condition; applying, using the plurality of features, a predictive model to a dataset representing the ground truth in relation to the defined target condition; determining, based on an output of the predictive model, one or more features of the plurality of features that are most relevant to the likelihood of the presence of the defined target condition; and determining, based on an output of the predictive model, a threshold value for each of the one or more features, beyond which the likelihood of the presence of the defined target condition is increased or decreased.

In some embodiments, the method may further comprise receiving an indication of a plurality of threshold values from which the threshold value is to be determined. The plurality of threshold values may comprise one or more quantiles of a frequency distribution of the dataset; and/or one or more user-defined thresholds.

The method may further comprise generating a risk assessment instrument including the determined one or more features and the determined threshold value for each of the determined one or more features; and providing the generated risk assessment instrument for presentation to a user.

In some embodiments, the method may further comprise determining a quality measure according to a quality metric for the output of the predictive model; and providing the quality measure for presentation to a user.

The method may, in some embodiments, further comprise modifying, responsive to a user input, the plurality of features to be analyzed; and applying the predictive model to the dataset using the modified plurality of features.

According to a third aspect, various embodiments provide a computer program product comprising a non-transitory computer readable medium, the computer readable medium having computer readable code embodied therein, the computer readable code being configured such that, on execution by a suitable computer or processor, the computer or processor is caused to perform any of the methods disclosed herein.

These and other aspects of the invention will be apparent from and elucidated with reference to the embodiments described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a simplified schematic of an example of an apparatus for determining features to be included in a risk assessment instrument according to various embodiments;

FIG. 2 is a flowchart of an example of a process of determining features to be included in a risk assessment instrument according to various embodiments;

FIG. 3 is an illustration of an example of a user interface according to various embodiments;

FIG. 4 is an illustration of a further example of a user interface according to various embodiments;

FIG. 5 is a flowchart of an example of a method for determining features to be included in a risk assessment instrument according to various embodiments;

FIG. 6 is a flowchart of a further example of a method for determining features to be included in a risk assessment instrument according to various embodiments; and

FIG. 7 is a simplified schematic of a computer-readable medium in communication with a processor.

### DETAILED DESCRIPTION OF EMBODIMENTS

Embodiments disclosed herein enable the determination of features to be included in a risk assessment instrument for estimating a risk or likelihood associated with a particular condition. For example, such an instrument may be used to estimate the likelihood of the presence of a particular target condition, or the likelihood of a particular target event occurring in the near term (e.g. on an intensive care unit, ICU) or in the distant future. While some examples disclosed herein are described in the field of clinical practice (e.g. estimating a likelihood of a subject suffering from of a particular medical condition), it will be understood that the principles disclosed herein may be applied in other settings including, but not limited to, for example, criminal and judicial sectors (e.g. calculating recidivism risk), machine maintenance, and financial industries (e.g. calculating behavior of financial instruments).

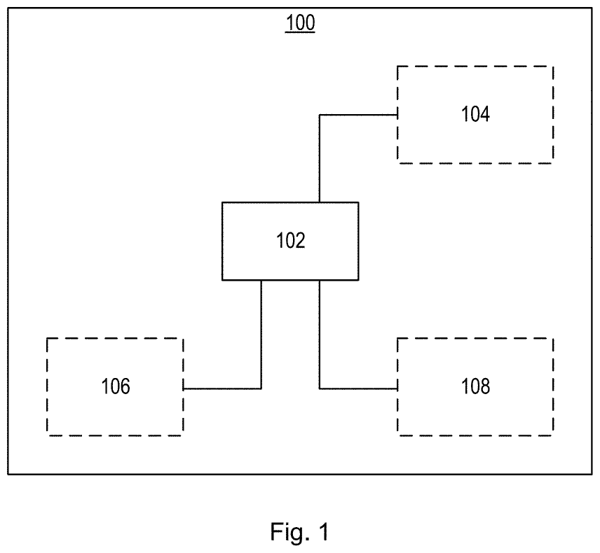

FIG. 1 shows a block diagram of an apparatus 100 that can be used for determining features to be included in a risk assessment instrument. With reference to FIG. 1, the apparatus 100 comprises a processor 102 that controls the operation of the apparatus 100 and that can implement the methods described herein. The apparatus 100 may further comprise a memory 106 comprising instruction data representing a set of instructions. The memory 106 may be configured to store the instruction data in the form of program code that can be executed by the processor 102 to perform the method described herein. In some implementations, the instruction data can comprise a plurality of software and/or hardware modules that are each configured to perform, or are for performing, individual or multiple steps of the method described herein. In some embodiments, the memory 106 may be part of a device that also comprises one or more other components of the apparatus 100 (for example, the processor 102 and/or one or more other components of the apparatus 100). In alternative embodiments, the memory 106 may be part of a separate device to the other components of the apparatus 100. For example, the apparatus 100 may be implemented as part of a cloud computing environment.

The processor 102 of the apparatus 100 can be configured to communicate with the memory 106 to execute the set of instructions. The set of instructions, when executed by the processor may cause the processor to perform steps of the methods described herein. The processor 102 can comprise one or more processors, processing units, multi-core processors or modules that are configured or programmed to control the apparatus 100 in the manner described herein. In some implementations, for example, the processor 102 may comprise a plurality of processors, processing units, multi-core processors and/or modules configured for distributed processing. It will be appreciated by a person skilled in the art that such processors, processing units, multi-core processors and/or modules may be located in different locations and may each perform different steps and/or different parts of a single step of the methods described herein.

In some embodiments, as illustrated in FIG. 1, the apparatus 100 may comprise at least one user interface 104 configured to receive any of the user inputs described herein. The user interface 104 may allow a user of the apparatus 100 to manually enter instructions, data, or information relating to the method described herein. For example, a user interface 104 may be used by a user (e.g. a clinician) to provide an indication of a feature or a plurality of features to be analyzed or included in a risk assessment instrument. In some embodiments, the user interface 104 may be used to present a risk assessment instrument to a user. In other embodiments, the user interface 104 may be used by a user to input data, such as thresholds to be included in the risk assessment instrument. The user interface 104 may be any type of user interface that enables a user of the apparatus 100 to provide a user input, interact with and/or control the apparatus 100. For example, the user interface 104 may comprise one or more switches, one or more buttons, a keypad, a keyboard, a mouse, a touch screen, a microphone or an application (for example, on a tablet or smartphone), or any other user interface, or combination of user interfaces that enables the user to provide data to the apparatus and/or via which the user can consume information from the apparatus 100.

In some embodiments, the user interface 104 (or another user interface of the apparatus 100) may enable rendering (or output or display) of information, data or signals to a user of the apparatus 100. As such, a user interface 104 may be for use in providing a user of the apparatus 100 (e.g. a designer of a risk assessment instrument or any other user) with information relating to or resulting from the method according to embodiments herein. The processor 102 may be configured to control one or more user interfaces 104 to provide information resulting from the method according to embodiments described herein. For example, the processor 102 may be configured to control one or more user interfaces 104 to render (or output or display) data (e.g. the risk assessment instrument, or features and/or thresholds to be included in such an instrument) using the methods described herein and/or any other outputs of the methods described herein. The user interface 104 may, in some embodiments, comprise a display screen, a graphical user interface (GUI) or other visual rendering component, one or more speakers, one or more microphones or any other audio component, one or more lights, a component for providing tactile feedback (e.g. a vibration function), or any other user interface, or combination of user interfaces for providing information relating to, or resulting from the method, to the user. In some embodiments, the user interface 104 may be part of a device that also comprises one or more other components of the apparatus 100 (for example, the processor 102, the memory 106 and/or one or more other components of the apparatus 100). In alternative embodiments, the user interface 104 may be part of a separate device to the other components of the apparatus 100.

In some embodiments, as illustrated in FIG. 1, the apparatus 100 may also comprise a communications interface (or circuitry) 108 for enabling the apparatus 100 to communicate with any interfaces, memories and devices that are internal or external to the apparatus 100. The communications interface 108 may communicate with any interfaces, memories and devices wirelessly or via a wired connection.

It will be appreciated that FIG. 1 shows the components required to illustrate this aspect of the disclosure and, in a practical implementation, the apparatus 100 may comprise other components in addition to those shown. For example, the apparatus 100 may comprise a battery or other power supply for powering the apparatus 100 or means for connecting the apparatus 100 to a mains power supply.

As noted above, the processor 102 is configured to perform steps of the methods described herein. In some embodiments, a memory (e.g. the memory 106) may be configured to store a set of instructions which, when executed by the processor 102 of the apparatus 100, cause the processor 102 to perform steps or functions as discussed below. The processor 102 is configured to receive an indication of a plurality of features to be analyzed, each feature of the plurality of features being potentially relevant to a likelihood of the presence of a defined target condition. The indication of the plurality of features to be analyzed may be received, for example, from a user input provided via the user interface 104. In other examples, the indication of the plurality of features may have been provided (e.g. by a user) at an earlier time and stored in a storage medium (e.g. the memory 106). Thus, the processor 102 may receive the indication of the plurality of features from the storage medium.

The received indication of the plurality of features are to be analyzed using the processor 102 as discussed below, so that the most appropriate feature or features of the plurality of features (i.e. the feature or feature most relevant to the target condition being assessed) can be included in the risk assessment instrument. As noted above, in the context of the present disclosure, a feature is a variable that is taken into account when assessing a particular risk or likelihood. The plurality of features may be provided by a user, for example based on the user's knowledge and experience. In other examples, a user may select the plurality of features from a list of features proposed or suggested by the apparatus 100. In yet other examples, the plurality of features may be provided entirely by the apparatus 100. For example, the plurality of features may be based on a set of features used previously, and known to be relevant to the particular risk or likelihood on which the risk assessment instrument is based.

The processor 102 is also configured to apply, using the plurality of features, a predictive model to a dataset representing the ground truth in relation to the defined target condition. The dataset may, for example, configure a set of data for which the presence or absence of the defined target condition is known. Such data is referred to as representing the ground truth, and this data is used by a predictive model to determine which of the plurality of features are considered relevant to (e.g. responsible for) the presence or absence of the defined target condition. For example, when determining a set of features to be included in a risk assessment instrument for determining the likelihood that a subject is suffering from Alzheimer's disease, a predictive model may be applied to a set of data relating to subjects for whom it is known whether each subject is suffering from Alzheimer's disease or not. The predictive model analyzes the ground truth data and looks for indications that any of the plurality of features is responsible for or contributes to the presence of Alzheimer's disease in a subject.

The predictive model applied by the processor 102 may be any predictive model suitable for analyzing a dataset representing the ground truth. The predictive model may, for example, comprise a machine learning model or algorithm. In some embodiments, the predictive model may comprise a logistic regression model. A logistic regression model measures the relationship between variables (i.e. features) by estimating probabilities using a logistic function, and the functionality of such a model will be familiar to those skilled in the art of machine learning and/or statistical analysis. In some embodiments, the predictive model parameterized as an integer programming problem where, contrary to traditional logistic regression models, the predictive model (e.g. a machine learning model) can be applied subject to user-defined constraints. In this way, the predictive model can be configured to output a feature/threshold combination from a single binarized feature set. By parameterizing the predictive model as an integer programming problem, all of the variables are restricted to be integers, and the output of the predictive model comprises an integer value or integer values. Dealing with integer values in a risk assessment instrument is beneficial as the task of calculating the risk assessment score is relatively straightforward.

As will be known by those skilled in the field of machine learning, the performance of a predictive model may be defined by a loss function (also referred to as a cost function). A loss function is a measure of how far away a particular solution is from an optimal solution to a problem being solved. A loss function for a logistic regression model can be said to be minimized when the model returns the optimal solution. The logistic loss function represents the accuracy of the predictive model, and sparsity of the predictive model is represented by the L0 norm regularization. According to one embodiment, the logistic loss function and the L0 norm regularization may be defined by:

$${min}_{λ ∈ A} \frac{1}{n} {\sum }_{i = 1}^{n} log ( 1 + {e}^{- {x}^{T} λ} ) + {C}_{0} {‖ λ ‖}_{0} \qquad [ 1 ]$$

where λ∈A means that ∀j, λⱼ∈{−10, −9, . . . , 0, . . . , 9, 10}, and where λ are the coefficients corresponding to the binarized features x.

The first term in equation [1] deals with the logistic loss (or cross entropy loss), while the second term deals with the model size, limiting the number of non-zero coefficients to be small.

The processor 102 is further configured to determine, based on an output of the predictive model, one or more features of the plurality of features that are most relevant to the likelihood of the presence of the defined target condition. Thus, by analyzing the data representing the ground truth, the predictive model can determine which of the received plurality of features are relevant to the likelihood of the presence of the defined target condition. In another way, the predictive model determines which of the plurality of features is/are most responsible for the presence of the defined target condition in a particular subject. Taking the Alzheimer's disease example, the predictive model may determine from the set of data representing the ground truth that the age of a subject is particularly relevant when determining risk/likelihood that a subject is suffering from, or will suffer from, Alzheimer's disease. Therefore, age may be determined to be a feature relevant to the likelihood of the presence of the defined target condition (i.e. Alzheimer's disease) in a subject.

In some examples, the processor 102 may be configured to receive an indication of maximum and/or minimum number of features to be included in the risk assessment instrument. For example, a user may provide an indication that a total of five features should be included in the risk assessment instrument. If a limit of the number of features to be included in the risk assessment instrument is provided, then the processor 102 may carry out its analysis of the plurality of features taking the indicated limit into account, such that the number of features determined to be most relevant to the likelihood of the presence of the defined target condition or within the defined limits.

The processor 102 is further configured to determine, based on an output of the predictive model, a threshold value for each of the one or more features, beyond which the likelihood of the presence of the defined target condition is increased or decreased. Thus, in addition to determining one or more features that are relevant to the likelihood presence of the defined target condition, the processor 102 determines, for each feature, a threshold value to be used in the risk assessment instrument. As with the determination of the relevant features, the predictive model is used to determine an appropriate threshold value for each feature, at (or beyond) which the likelihood of the presence of the defined target condition is even more relevant. For example, by analyzing the dataset representing the ground truth, the predictive model may determine a particular threshold value beyond which the likelihood of the presence of the defined target condition increases, decreases or is particularly relevant. For example, the likelihood of a subject contracting one medical condition may increase if the subject is over 70, but the likelihood of a subject contracting another medical condition may decrease if the subject is over 18 (e.g. not a child). The processor 102 may, therefore, determine that this threshold is the most relevant threshold concerning the feature, and that threshold value may be appropriate to include in the risk assessment instrument.

Considering again the Alzheimer's disease example, the predictive model, when applied to the dataset representing the ground truth, may determine that the likelihood of a subject suffering from Alzheimer's disease increases significantly the subject is over 68 years old. Therefore, if ‘age’ is determined to be a relevant feature when considering the likelihood of the presence of Alzheimer's disease, then a threshold value of 68 years may be determined for that feature, and used as the threshold value for the ‘age’ feature in the risk assessment instrument.

It will be apparent that a threshold value is determinable for a feature if the feature can be expressed or measured numerically. For example, if the processor 102 determines that the volume of a particular anatomical structure in a subject's body is of particular relevance to the likelihood of the presence of a defined target condition, then a threshold value may comprise a defined volume (e.g. in cubic centimeters). In another example, if the processor 102 determines that a particular score on the Alzheimer's Disease Assessment Scale (ADAS) is particularly relevant to the likelihood of the presence of a defined target condition (e.g. Alzheimer's disease), the threshold value may comprise a particular numerical value on that scale.

In the examples described above, the processor 102 determines absolute threshold values for features which are considered relevant in determining the likelihood of the presence of the defined target condition. However, in some embodiments, the processor 102 may determine that the relevant threshold value comprises a cut point dividing a frequency distribution of observations, referred to as a quantile. As will be familiar to those skilled in the art, the single cut point of the frequency distribution divided into two is the median, the two cut points of the frequency distribution divided into three are referred to as terciles (or tertiles) (i.e. the first tercile at the ⅓ point and the second tercile at the ⅔ point), the three cut points of the frequency distribution divided into four are referred to as quartiles (i.e. the first quartile at the ¼ point, the median and the third quartile at the ¾ point), the four cut points of the frequency distribution divided into five are referred to as quintiles (i.e. the first quintile at the ⅕ point, the second quintile at the ⅖ point, the third quintile at the ⅗ point and the fourth quintile at the ⅘ point), and so on. For example, the processor 102 may determine that the likelihood of the presence of a defined target condition increases significantly beyond the third quartile and, therefore, the processor may determine that the threshold value for a particular feature is the third quartile. Thus, in the Alzheimer's disease example, the processor 102 may determine from the dataset representing the ground truth that those subjects of an age exceeding the third quartile of the frequency distribution are at greater risk of suffering from Alzheimer's disease and, therefore, the value representing the third quartile may be chosen as a threshold value for that feature (i.e. age). In another, more general example, if the predictive model were to determine that division of the frequency distribution by quartiles provides the optimum solution, then all three thresholds (the first quartile, the median and the third quartile) may be used as thresholds for that feature (i.e. age), and one or more thresholds for a single feature may be included in the resulting risk assessment instrument.

The choice of which cut point to use as a threshold may depend on the form of the frequency distribution which may, for example, be unimodal (e.g. normal or skew normal), bimodal (e.g. symmetric or non-symmetric), and so on.

The frequency distribution can be represented in terms of a series of binarized statements for each feature or variable. For example, for a variable, Var1, a distribution may be represented in terms of three quantiles (i.e. the first quartile, the median and the third quartile):

1) Var1<First quartile of Var1

2) Var1≥First quartile of Var1

3) Var1≥Median of Var1

4) Var1≥Third quartile of Var1

Alternatively, the distribution may be represented in terms of four quintiles (i.e. 20, 40, 60, and 80 percentiles):

1) Var1<20-percentile of Var1

2) Var1≥20-percentile of Var1

3) Var1≥40-percentile of Var1

4) Var1≥60-percentile of Var1

5) Var1≥80-percentile of Var1

In existing systems, the choice of a particular cut point of the frequency distribution to use as a threshold value may be chosen by a user, based on past experience and/or based on a knowledge that values exceeding a particular threshold value are particularly relevant to the likelihood of the presence of a particular defined target condition. However, as noted previously, by selecting a particular cut point (e.g. a particular quantile) and omitting other cut points, there is a chance that relevant threshold values may not be taken into account. Some embodiments of the present disclosure avoid such omissions by considering multiple possible cut points (i.e. multiple quantiles) with regard to the ground truth dataset, in order to determine which, if any, quantile represents the most relevant threshold value.

The greatest understanding of which quantile represents the most relevant threshold value can be achieved by considering all possible quantiles (e.g., the median, the terciles, the quartiles, the quintiles, and so on), and determining which cut point represents the most relevant threshold value for inclusion in the risk assessment instrument. However, using the predictive model to consider all possible quantiles would be very processor-intensive and time-consuming. Therefore, according to some embodiments, the processor 102 may determine the threshold value for a feature by considering just a subset of quantiles (e.g., the median, the terciles and the quartiles). In this way, the multiple quantiles are taken into consideration, without requiring too much in terms of processing resources. The subset of quantiles to be considered by the processor 102 may, in some embodiments, be selected by a user, for example via the user interface 104. In this way, if a user has knowledge that a particular quantile might represent a relevant threshold value, they can choose to include that quantile in the subset of quantiles to be considered when the predictive model is applied to the ground truth dataset.

Considering various thresholds based on multiple quantiles may be considered to be a data-driven approach, or a statistical approach. In addition to such data-driven considerations, it may be beneficial for the processor 102 to take into account one or more user-defined thresholds. For example, the user may have some particular domain knowledge suggesting that the likelihood of the presence of a defined time condition increases at a particular threshold. Such user-defined thresholds may also be defined based on well-known or documented data, for example. According to some embodiments of the present disclosure, a user-defined threshold may be provided by a user (e.g. via the user interface 104). Any user-defined thresholds may be taken into account along with any data-driven considerations (e.g. the subset of quantiles) when the predictive model is applied to the ground truth dataset. In this way, the predictive model can consider all possible relevant thresholds, and can output the threshold value that it determines to be most relevant for inclusion in the risk assessment instrument.

In an example where the feature, or variable, is the age of a subject, a user may apply their knowledge and expertise to binarize the variable. For example, if the user believes that ‘age’ needs to be binarized based on the three thresholds of 60; 65; and 72 (e.g. because these particular ages are considered particularly relevant to the likelihood of the presence of the defined target condition), then the following binarizations may be applied:

1) Age <60

2) Age ≥60

3) Age ≥65

4) Age ≥72

Since each variable/feature has its own form of distribution and its own effect on the outcome of the predictive model, various data-driven and knowledge-based thresholds may be considered to achieve the best performance of the predictive model. Thus, in some embodiments, the determined threshold value may comprise a threshold value selected from a group of threshold values including: a quantile of a frequency distribution of the dataset; and a user-defined threshold.

Once the processor 102 has determined the feature(s) and corresponding threshold value(s), the risk assessment instrument may be generated. Thus, in some embodiments, the processor 102 may be further configured to generate a risk assessment instrument including the determined one or more features and the determined threshold value for each of the determined one or more features. For example, the processor 102 may provide the determined feature(s) and corresponding threshold value(s) for presentation to a user, for example via the user interface 104.

It may be beneficial for a user to know the accuracy of the output of the predictive model applied by the processor 102. Accordingly, in some embodiments, the processor 102 may be configured to calculate a quality measure according to a quality metric for the output of the predictive model. Numerous quality metrics suitable for assessing the output of a predictive model will be familiar to those skilled in the art. In some embodiments, the area under a receiver operating characteristic curve (also referred to as the area under an ROC curve, or simply AUC) may be calculated by the processor 102, and used as a quality measure for the output of the predictive model. In some embodiments, a calibration error may be calculated by the processor 102, and used as a quality measure for the output of the predictive model. In other embodiments, multiple quality measures may be calculated.

In some embodiments, one or more quality measures may be presented to a user (e.g. via the user interface 104) along with the outputs of the predictive model (e.g. the determined features and the determined thresholds) so that the user can see how the outputs of the predictive model fair regarding the quality metric(s). In this way, a user can tell whether or not an output of the predictive model meets a defined quality standard. If the output of the predictive model does meet a required standard (e.g. if the to measure meets or exceeds a defined quality threshold value), then the user may trust the output and accept the output for use in the risk assessment instrument. However, if the quality measure of the output of the predictive model does not meet a required standard (e.g. if the quality measure falls below a defined quality threshold value), then the user may choose not to use the output in the risk assessment instrument, and may choose to adjust one or more parameters of the predictive model so as to improve the quality measure of the output.

In some embodiments, the processor 102 may be configured to take action if it is determined that the calculated quality measure is below a defined quality measure threshold. In one example, the quality measure may fall below a defined quality measure threshold if the plurality of features used by the predictive model fails to include a sufficient number of relevant features relevant to the likelihood of the presence of the defined target condition. In other words, the processor 102 may determine that, using the particular combination of the plurality of features, the predictive model cannot accurately predict the likelihood of the presence of the defined target condition. A more accurate prediction of the likelihood may be achieved if a greater number of features (or a selection of more relevant features) is used by the predictive model. Thus, in some embodiments, the processor 102 may be configured to reapply the predictive model using a greater number of features or a revised set of features.

In one embodiment, responsive to determining that the calculated quality measure is below a defined quality measure threshold, the processor 102 may be configured to add a feature to the plurality of features to obtain a revised feature set. The feature to be added to the plurality of features may be selected automatically by the processor 102 based on one or more rules, or may be selected by a user. The processor 102 may then apply, using the revised feature set, the predictive model to the dataset. The processor 102 may then determine, based on an output of the predictive model, one or more features of the revised feature set that are most relevant to the likelihood of the presence of the defined target condition. The processor 102 may then determine, based on an output of the predictive model, a threshold value for each of the one or more features, beyond which the likelihood of the presence of the defined target condition is increased or decreased. The processor 102 may then determine a revised quality measure according to the quality metric for the output of the predictive model. In other words, once a revised feature set has been obtained by adding a feature to the initial plurality of features used by the predictive model, the predictive model can be reapplied to obtain new outputs (i.e. one or more features and corresponding threshold values) along with a corresponding quality measure. If the revised quality measure meets or exceeds the defined quality measure threshold, then the new outputs may be accepted and used in the risk assessment instrument. Otherwise, the processor 102 may add a further feature to the plurality of features and reapply the predictive model again. This process may be repeated until a required quality measure is achieved.

As noted above, the user interface 104 may be used by a user to input data to be used by the apparatus 100/processor 102 and/or to view or receive information outputted by the apparatus 100/processor 102. In some embodiments, user interface 104 may be configured for receiving from a user the indication of the plurality of features to be analyzed and/or an indication of a plurality of threshold values from which the threshold value for each of the one or more features is to be determined. For example, a user may input a plurality of features manually, for example using a keyboard, or by selecting a plurality of features from a list. The indication of a plurality of threshold values may be provided by a user in a similar way, for example by selecting the threshold values from a list. The user may, for example, input one or more threshold values known or expected to be relevant based on their domain knowledge and/or an indication of one or more quantiles which should be included in the assessment made using the predictive model.

The user interface 104 may, in some embodiments, be further configured for presenting to a user an indication of the determined one or more features and an indication of the determined threshold for each of the one or more features. In some embodiments, the user interface 104 may further be for presenting to a user a graphical representation of the determined one or more features and an indication of the determined threshold for each of the one or more features. Thus, the indication may comprise a graphical representation.

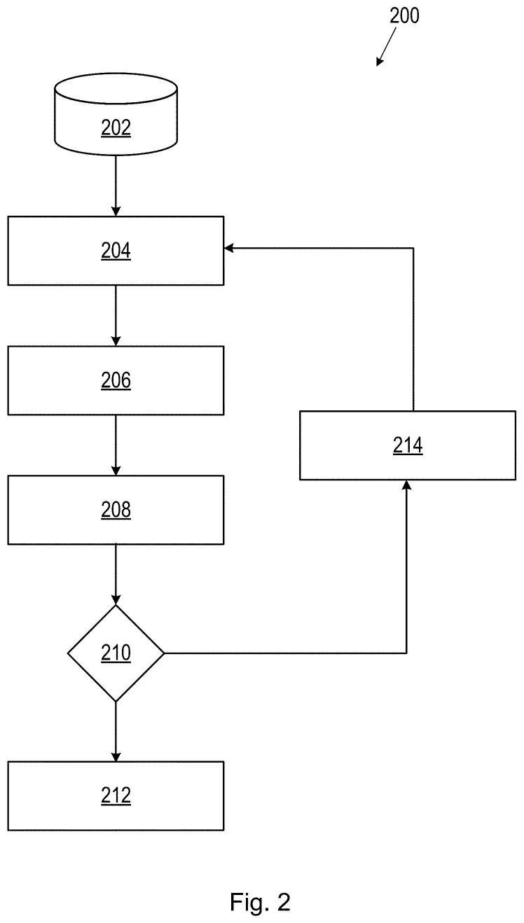

FIG. 2 is a flowchart of an example of a process 200 of determining features to be included in a risk assessment instrument according to various embodiments. Specifically, FIG. 2 outlines various functions performed by the apparatus 100. Various data may be stored in a database 202 and acquired by the processor 102. For example, the database 202 may store indications of a plurality of features relevant to the likelihood of the presence of one or more target conditions. Similarly, the database 202 may store indications of threshold values for one or more of the features. The database 202 may also store data representing the ground truth in relation to one or more target conditions. At block 204, the indication of the plurality of features to be analyzed may be received, for example via the user interface 104. The predictive model is applied at block 206, for example to a dataset representing the ground truth in relation to a target condition. Once the predictive model has been applied in 206, a quality measure may be calculated at block 208. At block 210, a check may be made regarding whether or not the output of the predictive model is satisfactory (e.g. whether or not the quality measure calculated in block 208 meets or exceeds a threshold level. If the quality check at block 210 is successful, then the process proceeds to block 212, where the outputs of the predictive model are presented to a user, for example in the form of a risk assessment instrument. If, however, the quality check at block 210 is unsuccessful, then the process may proceed to block 214, where one or more constraints or parameters of the predictive model may be altered. For example, an additional feature maybe added to the plurality of features to be analyzed. The additional feature may be added by a user, at block 204, automatically by the processor 102, such that the process can proceed straight to applying the predictive model at block 206. In some examples, the results of the quality check at block 208 may be presented to the user (e.g. via the user interface 104) at block 212. Upon reviewing the calculated quality measure and the outputs of the predictive model, a user may choose to adjust one or more of the constraints or parameters of the predictive model, such that the process may proceed from block 212 to 214.

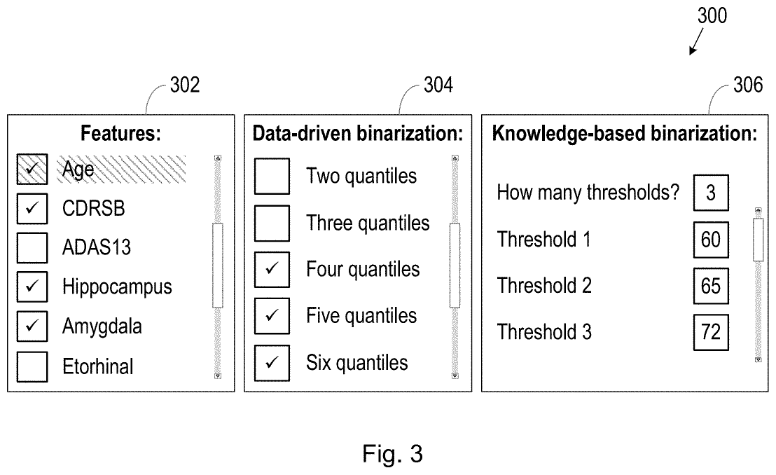

An example of a user interface 300 for use in various embodiments of the present disclosure is shown in FIG. 3. The example user interface 300 includes a first column 302 enabling a user to input or select one or more features to be analyzed, a second column 304 enabling a user to input or select data-driven binarization options, and a third column 306 enabling a user to input or select knowledge-based binarization options. In this example, the first column 302 includes a list of possible options for use by the predictive model. A user may select those features which are considered likely to be relevant to the likelihood of the presence of the target condition being investigated such as, for example, age, CDRSB (Clinical Dementia Rating Some of the Boxes), ADAS13 (Alzheimer's Disease Assessment Scale), hippocampus (i.e. the volume of a subject's hippocampus), amygdala (i.e. the volume of a subject's amygdala), and etorhinal (i.e. the volume of a subject's etorhinal cortex). In one embodiment, a user may tick or otherwise select boxes associated with those features that are to be included. The second column 304 includes a list of possible binarization options to the considered by the predictive model. In this example, the second column 304 includes the options of two, three, four, five and six quantiles, though other quantiles may be included. Again, a user may select boxes associated with those binarization options that are to be considered by the predictive model. The third column 306 includes a list of possible knowledge-based thresholds be considered by the predictive model. In this example, a user may enter the number of thresholds to be considered, and a threshold value for each threshold.

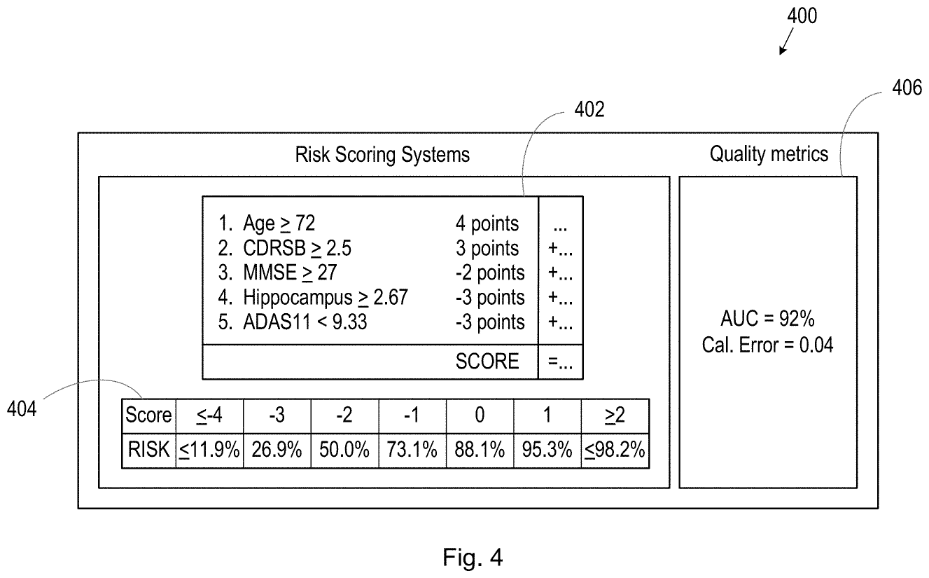

FIG. 4 shows a further example of a user interface 400 for use in various embodiments of the present disclosure. In FIG. 4, the user interface 400 shows an example of a risk assessment instrument generated based on the options shown in the example of FIG. 3. In this example, the predictive model has determined, based on the selections shown in FIG. 3, the most relevant features for determining the likelihood of the presence of a particular target condition (i.e. Alzheimer's disease in the examples shown in FIGS. 3 and 4), and the most relevant threshold value for each feature. In the user interface 400, the features and their corresponding threshold values are shown in a block 402. In this example, the relevant features and thresholds are:

Age ≥72;

CDRSB≥2.5;

MMSE (Mini-Mental State Examination Score)≥27;

Hippocampus volume ≥2.67;

ADAS11<9.33.

A feature score (i.e. a number of points) is associated with or assigned to each feature, such that, if a subject meets the criteria for a particular feature, the associated score is awarded. Methods for calculating and assigning feature scores to features in the context of risk assessment instruments will be known to those skilled in the art.

According to one example, based on the feature score assigned to each feature, a total score may be calculated by:

score=4×(Age ≥72)+3×(CDRSB≥2.5)−2×(MMSE≥27)−3×(Hippocampus≥2.67)−3×(ADAS11<9.33) [2]

A risk or likelihood of developing Alzheimer's disease may therefore be calculated by:

$$Risk = \frac{1}{1 + exp ( - score - 2 )} \qquad [ 3 ]$$

In the user interface 400, a guide to the risk associated with various scores is shown in block 404, so that a user can quickly determine a subject's risk of developing Alzheimer's disease based on the score they achieved using the risk assessment instrument. The user interface 400 also includes a block 406, which displays various quality measures calculated according to various quality metrics. In this example, the block 406 shows an Area Under the Curve (AUC) value of 92% and a calibration error value of 0.04. From these values, a user can quickly determine the accuracy of the outputs from the predictive model.

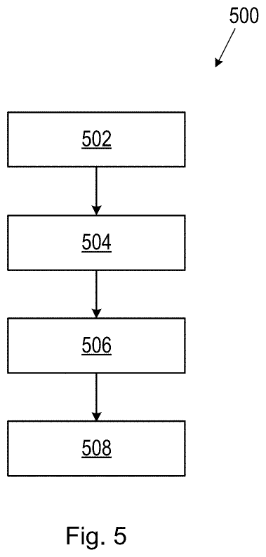

FIG. 5 is a flowchart of an example of a method 500 for determining features to be included in a risk assessment instrument according to various embodiments. The method 500 may, for example, be performed using the apparatus 100. One or more steps of the method 500 may be performed using the processor 102. The method 500 comprises, at step 502, receiving an indication of a plurality of features to be analyzed, each feature of the plurality of features being potentially relevant to a likelihood of the presence of a defined target condition. At step 504, the method 500 comprises applying, using the plurality of features, a predictive model to a dataset representing the ground truth in relation to the defined target condition. The method 500 comprises, at step 506, determining, based on an output of the predictive model, one or more features of the plurality of features that are most relevant to the likelihood of the presence of the defined target condition. At step 508, the method 500 comprises determining, based on an output of the predictive model, a threshold value for each of the one or more features, beyond which the likelihood of the presence of the defined target condition is increased or decreased.

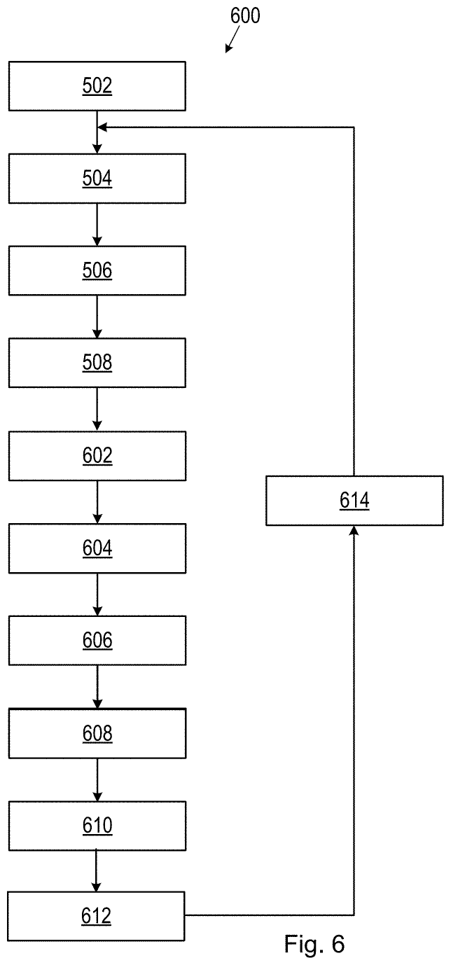

FIG. 6 is a flowchart of a further example of a method 600 for determining features to be included in a risk assessment instrument according to various embodiments. The method 600 may include one or more features of the method 500. The method 600 may further comprise, at step 602, receiving an indication of a plurality of threshold values from which the threshold value is to be determined. The indication of the threshold values may, for example, be provided by a user (e.g. via the user interface 104). The plurality of threshold values may comprise one or more quantiles of a frequency distribution of the dataset and/or one or more user-defined thresholds.

At step 604, the method 600 may further comprise generating a risk assessment instrument including the determined one or more features and the determined threshold value for each of the determined one or more features. The risk assessment instrument may, for example, comprise an instrument as shown in the example of FIG. 4. The method 600 may further comprise, at step 606, providing the generated risk assessment instrument for presentation to a user. For example, the risk assessment instrument may be displayed via the user interface 104.

The method 600 may further comprise, at step 608, determining a quality measure according to a quality metric for the output of the predictive model. As discussed above, the quality metric used for determining the quality of the predictive model may, in some embodiments, comprise an area under the curve measurement and/or a calibration error measurement. At step 610, the method 600 may further comprise providing the quality measure for presentation to a user. Again, the quality measure may be displayed to a user using the user interface 104. In one example, the quality measure may be displayed as shown in the example of FIG. 4.

In some embodiments, it may be desirable to modify the plurality of features to be analyzed. For example, if the quality measure falls below a defined quality threshold value, entry applying the predictive model using a modified set of features may result in an improved quality measure. The plurality of features may be modified automatically, for example by the processor 102, or manually, for example by a user. The user may make such a modification via the user interface 104. Thus, the method 600 may comprise, at step 612, modifying, responsive to a user input, the plurality of features to be analyzed. At step 614, the method 600 may comprise applying the predictive model to the dataset using the modified plurality of features. Thus, once the plurality of features have been modified, the method may revert to step 504, at which the predictive model is applied. In some embodiments, the process of repeating the application of the predictive model using a modified plurality of features may be repeated until an end criterion is met (e.g. until the calculated quality measure meets or exceeds a defined quality threshold value).

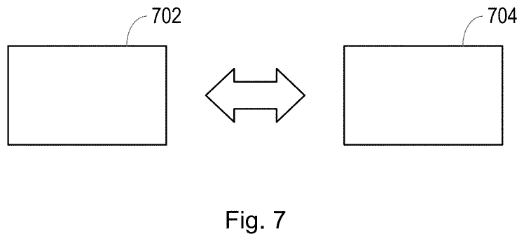

FIG. 7 is a simplified schematic of a processor 702 in communication with a computer-readable medium 704. According to one aspect, the present disclosure provides a computer program product comprising a non-transitory computer-readable medium 704, the computer-readable medium having computer-readable code embodied therein, the computer-readable code being configured such that, on execution by a suitable computer or processor 702, the computer or processor is caused to perform steps of the methods 500, 600 disclosed herein. The processor 702 may, in some embodiments, comprise or be similar to the processor 102 discussed above.

The processor 102, 702 can comprise one or more processors, processing units, multi-core processors or modules that are configured or programmed to control the apparatus 100 in the manner described herein. In particular implementations, the processor 102, 702 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus for determining features to be included in a risk assessment instrument, the apparatus comprising:
a processor configured to:
receive an indication of a plurality of features to be analyzed, each feature of the plurality of features being potentially relevant to a likelihood of the presence of a defined target condition;
apply, using the plurality of features, a predictive model to a dataset representing the ground truth in relation to the defined target condition;
determine, based on an output of the predictive model, one or more features of the plurality of features that are most relevant to the likelihood of the presence of the defined target condition; and
determine, based on an output of the predictive model, a threshold value for each of the one or more features, beyond which the likelihood of the presence of the defined target condition is increased or decreased.

2. An apparatus according to claim 1, wherein the determined threshold value comprises a threshold value selected from a group of threshold values including: a quantile of a frequency distribution of the dataset; and a user-defined threshold.

3. An apparatus according to claim 1, wherein the processor is further configured to:
generate a risk assessment instrument including the determined one or more features and the determined threshold value for each of the determined one or more features.

4. An apparatus according to claim 1, wherein the predictive model comprises a logistic regression model.

5. An apparatus according to claim 1, wherein the predictive model applied to the dataset is parameterized as an integer programming problem.

6. An apparatus according to claim 1, wherein the processor is configured to:
calculate a quality measure according to a quality metric for the output of the predictive model.

7. An apparatus according to claim 6, wherein the processor is configured, responsive to determining that the calculated quality measure is below a defined quality measure threshold, to:
add a feature to the plurality of features to obtain a revised feature set;
apply, using the revised feature set, the predictive model to the dataset;
determine, based on an output of the predictive model, one or more features of the revised feature set that are most relevant to the likelihood of the presence of the defined target condition;
determine, based on an output of the predictive model, a threshold value for each of the one or more features, beyond which the likelihood of the presence of the defined target condition is increased or decreased; and
determine a revised quality measure according to the quality metric for the output of the predictive model.

8. An apparatus according to claim 1, further comprising:
a user interface for receiving from a user the indication of the plurality of features to be analyzed and/or an indication of a plurality of threshold values from which the threshold value for each of the one or more features is to be determined.

9. An apparatus according to claim 8, wherein the user interface is further for presenting to a user an indication of the determined one or more features and an indication of the determined threshold for each of the one or more features.

10. A method for determining features to be included in a risk assessment instrument, the method comprising:
receiving an indication of a plurality of features to be analyzed, each feature of the plurality of features being potentially relevant to a likelihood of the presence of a defined target condition;
applying, using the plurality of features, a predictive model to a dataset representing the ground truth in relation to the defined target condition;
determining, based on an output of the predictive model, one or more features of the plurality of features that are most relevant to the likelihood of the presence of the defined target condition; and
determining, based on an output of the predictive model, a threshold value for each of the one or more features, beyond which the likelihood of the presence of the defined target condition is increased or decreased.

11. A method according to claim 10, further comprising:
receiving an indication of a plurality of threshold values from which the threshold value is to be determined;
wherein the plurality of threshold values comprises one or more quantiles of a frequency distribution of the dataset; and/or one or more user-defined thresholds.

12. A method according to claim 10 or claim 11, further comprising:
generating a risk assessment instrument including the determined one or more features and the determined threshold value for each of the determined one or more features; and
providing the generated risk assessment instrument for presentation to a user.

13. A method according to claim 10, further comprising:
determining a quality measure according to a quality metric for the output of the predictive model; and
providing the quality measure for presentation to a user.

14. A method according to claim 10, further comprising:
modifying, responsive to a user input, the plurality of features to be analyzed; and
applying the predictive model to the dataset using the modified plurality of features.

15. A computer program product comprising a non-transitory computer-readable medium, the computer-readable medium having computer-readable code embodied therein, the computer-readable code being configured such that, on execution by a suitable computer or processor, the computer or processor is caused to perform the method of claim 10.
